(19) United States
(12) Patent Application Publication
(10) Pub. No.: US 2024/0136035 A1
(43) Pub. Date: Apr. 25, 2024

(54) SYSTEM AND METHOD FOR NEURO-SENSORY BIOFEEDBACK ARTIFICIAL INTELLIGENCE BASED WELLNESS THERAPY

(71) Applicant: Alexander Socransky, Los Angeles, CA (US)

(72) Inventor: Alexander Socransky, Los Angeles, CA (US)

(21) Appl. No.: 17/972,513

(22) Filed: Oct. 23, 2022

(51) Int. Cl. G16H 20/00; G16H 10/60; G16H 50/20; G16H 70/20; G16H 70/40; G16H 80/00

(52) U.S. Cl. CPC G16H 20/00; G16H 10/60; G16H 50/20; G16H 70/20; G16H 70/40; G16H 80/00

(57) ABSTRACT

The present invention pertains to neurosensory and physical sensor AI therapy through pattern recognizing, modeling, and suggestive technology. According to the invention an AI program enabled with facial recognition monitors and analyzes different sensors on a patient to discover what the best treatment plan for overall body wellness, psychological coping skill sets, meditation techniques, musical, visual, and auditory, supplement, drug, food, exercising, or other techniques. The same set of sensors may be used to assess a user's mental state and then generate and implement a series of exercises or steps geared to improve their overall mental state. These treatment plans will be based on the type of learning that the patient responds to best. Other biometrics are also measured, like lie detection and micro expressions. All these biometrics that are measured become reference points to track progress of the patient and the therapies and make predictive analysis for therapies.

### BACKGROUND OF THE INVENTION

The present invention is directed to overall wellness and cognitive therapy and mental health treatment.

Artificial Intelligence/neural networks and/or machine learning are to be thought of from this point forward as AI or Artificial Intelligence. Artificial Intelligence is the development of computer systems that are programmed to behave like humans and with human-like thinking. The main asset of AI is the ability to react and learn from its programming. With the use of AI, there is the ability for technology to reach further than humans can in thinking. There already exists AI technology in the treatment of certain diseases and conditions, like migraines or tinnitus, but there has yet to exist a place for AI in general holistic approach to the mind and body. AI allows these therapeutic practices to be constantly monitored and updated, taking away the need of a human, and allowing for more vulnerability and honesty. There also already exists technologies to study hormones and DNA, but the present invention will take these technologies to create a holistic AI health program.

### SUMMARY OF THE INVENTION

The present invention pertains to neurosensory and other biometric sensors AI therapy technology. According to the invention, an AI program with enabled facial recognition, monitors and analyzes different sensors on a patient to discover the best psychological and physiological treatment plan, whether that is coping skill sets, meditation techniques, musical, visual, and auditory input, supplements, exercise, even suggested drugs with a physicians consent and diagnosis of the data provided by the system or other activities or other techniques which have been proven through the patient's patterns in their sensor data. This ensures the desired outcome to obtain a certain flow state, as well as an overall suggested program of well-being. The same set of sensors may be used to assess a user's mental state and then generate and implement a series of exercises or steps geared to improve the overall mental state and state of well-being. These treatment plans will be based on the type of learning that the patient responds to best based on the recorded sensory data, correlated data, best known practices, known treatment plans, or recorded data sets of similar value from other users. Other biometrics are also measured, like lie detection, micro expressions, ECG, MCG, and vocal stress analysis. All these biometrics that are measured become reference points to track the progress of the patient and the therapies and are potentially correlated with similar value data points of similar patterns with other users.

Using a sweep of sensors such as phone laser/lidar allows the tracking of eye movements and facial micro-expressions. This would allow the user's phone camera to detect signs of stress in their face and eye movements and provide this biometric feedback to the AI. Since smartphones are touch and pressure sensitive, the phone can also collect haptic data from the user and emit haptic feedback as a way of both detecting stress in the user and relieving stress via haptic feedback. The phone camera as such can be used to analyze body language, while biometric sensors such as a Smart Watch or other device can measure oxygen and EEG, MCG, sweat preparation, glucose levels, heart rate analysis, alcohol levels, stress, etc. Analyzing voice stress using smart neuropsychology, along with measuring cortisol levels, vagus nerve activity, other cranial stimulation, or future physiological stimulation, other future brain sensors, and other sensors, can allow the AI to determine which therapeutic treatments are working and which are not working for the patient. All these AI technologies allow for the best-tailored system for the psychological and physical treatment of the patient.

Smartphones and other smart devices have reached a point of sophistication at which they know more about their user than anyone else. Photos, notes, documents, text messages, videos, social media engagement, media consumption, and more are all data collected on a user's phone that provides the device with information that no one else has access to. The present invention provides an opportunity for this data to be analyzed by AI with permission of the user and compared across users to determine patterns that may arise among specific individuals who are likely to commit crimes and acts of terror and violence and provide preventative therapies and treatments that are proven to provide comfort and counseling to the user and decrease the likelihood of them committing any acts of violence. In this way, the present invention can not only act as a personal health manager but as a public health and safety tool as well. Additionally, AI can determine (via facial recognition and other biosensors) if any media being consumed is triggering symptoms of anxiety, depression, or other mental health concerns for the user. This provides an opportunity to give the user insight into how the media they consume impacts their mental and physical health and provides the user with media alternatives or perhaps putting filters and control settings on what types of media are featured on the user's devices to prevent these negative encounters.

On the contrary, the present invention can also track the dopaminergic response to the media consumed by the user as well. While some media can be shown to trigger negative health responses such as anxiety and depressive thoughts, other media can be shown to have a positive impact on the user's mental and physical health. However, often times this positive response is not proportional to the activity, leading to excessive amounts of dopamine release triggered by media engagement. By learning these patterns for each user, AI can regulate the amount of media consumed which provides the user with the most dopamine or the amount of dopamine released when engaging with certain media, in order to avoid overstimulation and social media addiction. This can help users seek dopamine from more real-life experiences as they are not receiving overstimulating amounts of dopamine from their phones or other media-sharing devices. This can help users release more real-life, “earned” dopamine and motivate the user to participate in real-world activities that may be difficult for them in order to replenish the dopamine that they have grown accustomed to receiving from consuming media on their phone. Furthermore, the present invention can go as far as to modulate the video, audio, text, or other forms of media presented to the user in order to deter the user from consuming media that is known by the present invention to have a negative impact on the user's overall wellness.

With the use of facial recognition features through a phone's camera, different therapeutic techniques can be used based on the facial reactions of the user. AI can collect data on the user's stressed versus relaxed facial expressions to determine which unique set of techniques have the best effect on the user and promote the use of these therapies. However, facial recognition is just one of many ways for AI to determine the best therapeutic practices for the user. Additionally, devices like Fitbit, Smart Watches, and other devices can detect changes in cortisol levels, and quantum magnetometers and ECG can detect the heart's magnetic field. Additionally, the use of RFID supplements and DNA analyzers can help track a patient's medication and hormone levels. All this data can help determine which therapeutic techniques are producing the best holistic outcome.

Wearable medical devices such as ubiquitous sensors can allow for the incorporation of more biofeedback collected from the user throughout the day. Pulse oximeters, glucose monitors, and activity trackers can be utilized to help inform the user of the best management practices. Increased levels of cortisol due to stress can increase blood glucose levels, which can quickly become a health concern especially for diabetics. Glucose monitors can help provide feedback to the user, allowing them to make informed decisions about their activity and diet. By providing feedback to the user regarding the impact of their daily habits on their health, the user becomes motivated to maintain habits that are shown to be improving their mental and physical health. These devices allow the user to compete with themselves to form the best habits and look back at the progress that they have made. This allows the user to play a key role in their own treatment, providing them with a sense of reward and accomplishment along with increased health benefits and an overall sense of well-being.

Another technique the AI can use is vagus nerve stimulation. The vagus nerve is the tenth of the twelve cranial nerves and is responsible for parasympathetic functions including heart rate, digestion, and immune system function. Stimulating the vagus nerve activates the body's “rest and digest” mode, bringing down levels of stress and anxiety by lowering cortisol levels. Vagal nerve stimulators are an FDA-cleared form of treatment for depression, anxiety, and insomnia. This stimulation can increase the levels of serotonin in the brain and lower the levels of cortisol. If a user has low levels of serotonin and high levels of cortisol, the AI can monitor that via wearable biosensors and determine if this nerve stimulation will have a positive effect on the user. The AI can then “prescribe” vagus nerve stimulation sessions as treatment whenever high cortisol levels are detected. In addition to vagus nerve stimulation, a headband or other Bluetooth device that can stimulate and connect with EEG technology could be worn in order to scan cranial electrical activity in order to provide biofeedback on a neural level. Devices such as this have been used to promote meditation in order to improve emotional regulation and calmness. The EEG device could allow the AI to receive neural biofeedback while the user undergoes several different therapies such as listening to music or meditating in order to determine which provides the most calming effect on a neural level.

The functions in a human body are determined by a magnetic field and a set of patterns maintained through homeostasis. Different fluctuations of magnetic fields in different functions in the body have different effects on the body. Knowing the magnetic field at the resting state gives AI the ability to track any fluctuations that deter from the resting state. This gives power to the AI to detect these differences and know the cause of them and what needs to be done to fix them.

Furthermore, the use of electromagnetic, ultrasonic, and even light stimulation at specific frequencies has been shown in previous research to cause neurological changes on a chemical level. The therapeutic use of devices capable of emitting specific frequencies such as electromagnetics, ultrasonics, lasers at varied pulsations, or other mechanisms containing the ability to vibrate at a resonant frequency could be incorporated into the AI in order to generate a therapeutic chemical reaction. One such reaction can be induced via a dampened spring model induced by self-assembly nanoparticles for the enhancement or suppression of DNA in specific target regions in order to create changes in peptide levels which could positively impact an individual's mental and physical health. The ability to use frequencies in this manner can be used medically as well as for the design of various genetically modified items such as amino acids, peptides, and hormones as it has been shown that certain frequencies can accelerate proteolysis or the breakdown of proteins into peptides and amino acids via enzymes. With the use of synthesized bio-functional nanoparticles and high resonant frequency stimulating devices and ultrasound, ions and other particles and molecules can be vibrated into essential amino acids, which are the building blocks of proteins such as RNA and DNA. The ability to medically induce the creation of amino acids allows for a more immediate solution to hormone and nutritional imbalances.

A more advanced approach involving the use of a particle accelerator can alter the force gravitation which can increase the speed of a given system with varying changes in ionization, amino acid sequences, and peptides. One example of this could be the increased speed of the DNA cycle of transcription and translation in addition to changes in DNA enhancement and suppression as previously mentioned. One example in which these technologies could be applied by the AI as potential treatment at an electrochemical level is to increase serotonin or cortisol levels in response to a given set of recently observed biometrics, among other things.

Many mental disorders such as PTSD can alter the structure of an individual's DNA, presenting us with a biomarker for these disorders that, upon at-home DNA analysis, can be identified and treated according to that individual's unique case. DNA analysis in this manner can be combined with the use of bio-sensor data via wearable nanotechnology sensors and/or devices such as iWatches, Fitbits, and other devices in order to determine the best-fit diagnosis and the appropriate treatment based on similar data across user data. Current models of wearable nanotechnology have the ability to measure solute levels in sweat which can indicate stress and anxiety levels as well as physical health concerns such as hydration and electrolytic intake, all of which are important data for proper treatment upon AI analysis.

The AI can use certain ratios found in the recorded data to determine which therapies are working better than others for the user. Based on certain parameters, the AI can make these determinations. Adding these parameters into a database will also allow for the easy discovery of similar patterns across different users, increasing the speed and ease with which the right therapeutic technique for a user can be found. If in this database a user is found to have similar reactions to certain techniques compared to several other datasets, the AI can determine what worked for the first user and use this as a guide for determining the best therapy techniques for the second user, giving the second user a better and quicker chance at finding the perfect therapeutic techniques. All the collected data may be encrypted with the use of artificial neural networks in order to ensure patient privacy, a technique that has already been suggested for use in hospital data collection and highly sensitive personal information. The use of the neural network can allow the AI to submit all the data in an encrypted form to a cloud space or other methods of data storage such as a blockchain, regular servers, and others and likewise retrieve data from the cloud or other methods of storage such as a blockchain, regular servers, and others. in an encrypted form in order to make comparisons across the data while maintaining each user's privacy. This is one of many other methods of data storage that could be used by the present invention in order to ensure data security and privacy among users.

With the increased improvement in users' mental and bodily health, they are ultimately improving their physical health as well. As there is currently no database collecting information on the different types of trauma or other types of illness or wellness biomarkers people endure nor is there a database on the best therapeutic techniques for these traumas or wellness biomarkers, the present invention will create a space for all this information to be collected and easily accessed by the AI to determine the best-proven therapy for any given trauma or wellness. With this so-called “concierge mental and physiological manager,” the user will be able to easily express and diagnose their problems in a more convenient manner than by talking to a regular therapist, thus providing a holistic, proactive way of managing both physical and mental health.

The goal of the present invention is not to replace therapists, doctors or other medical professionals, which would all still be available, but rather provide a cost-effective alternative that takes a more holistic approach. Because good therapists and other healthcare professionals can be very expensive and hard to find, the present invention would make mental health and physical healthcare more available to the average person. The ability of the present invention to collect a variety of data in a non-invasive manner from the user's own home allows the AI to gain an understanding of the user's health much faster than traditional medicine, which often requires the advice of multiple specialists which can take months to book appointments with and large amounts of money in medical bills. The AI would even be able to monitor the intake of the user's prescription medication and supplements, warn against any potential chemical interactions across multiple medications, and monitor any changes in health. The present invention can even provide the healthcare professionals with suggestions in terms of tailoring prescription types and doses for mental health treatment. One example of this could be monitoring a user who takes serotonin inhibitors, and specifically monitoring their changes in hormones and behavior in response to the prescription. This information can then be reported to the individual's primary care provider. This system of medication monitoring can be applied to any type of medication or supplement.

Furthermore, the present invention has the ability to provide more preventative treatment than the reactive way in which the medical field currently operates. The use of several neurosensory and biometric sensors allows for a holistic preventative approach to medicine. As AI collects and analyzes data from these sensors, it gains insights into the user's health and habits that would otherwise go unnoticed by a medical professional until a visible problem presents itself. The ability of the AI to track and monitor the user's health and habits allows for it to integrate and prescribe various treatments and therapies to prevent any given habits from developing into a health concern. Should a concerning pattern of biometric data arise, the AI could even outsource to the patient's primary care provider or other medical professionals who can then outsource to a group of specialists, thus fully integrating the present invention into today's healthcare system. Additionally, if the present invention were to detect a health emergency from the various biosensors and biometric data provided, it could dial an emergency hotline such as 911 and contact emergency medical technicians (EMTs) in order to rapidly provide the necessary emergency care, and upon their arrival, provide the medical professionals with real-time health data related to the incident. To further the integration of the present invention with the healthcare system, insurance companies could offer discounts to individuals who use the present invention in order to encourage its implementation in the world of healthcare.

The present invention can go even further with new ideas of real-time analysis of DNA via monitoring the fluctuations of several biomarkers in the body and how different hormones and organs can activate or deactivate DNA and protein synthesis in various regions within the body given the right environment. This is a systemic approach to mental health, longevity, stress and best flow state. The present invention will monitor all these biomarkers and be able to observe the different patterns of various DNA strands with genome sequencing and the use of wearable hormone sensors, nanopores, and ionic changes in order to map alterations in the DNA and the different influences of mental treatments. The AI will monitor pharmaceuticals and the intake of supplements, food, alcohol, exercise, mental thoughts, even going to watch certain types of movies or to listen to certain types of audio, playing with your children or animals or taking a walk, etc. to see what is giving the user the best performance in their mental health and body performance or make suggestions and with prescribed medications being sent for approval from or suggested to their primary care provider. to look at it for further analysis. With the ability to track changes in DNA sequencing in real-time, the present invention will also contain the ability to monitor DNA for instances of bioterrorism. Should AI detect changes in DNA sequencing with signatures that match bioterrorism, or even any unhealthy or potentially harmful changes made to a DNA sequence, preserved backup DNA sequences and CRISPR technologies or other methods can change the altered DNA sequence back to a healthy code.

The monitoring of all these inputs can be used in a micro and/or macro scale. For example, in a micro scenario, there is a catastrophe, and your biometric sensors can see you're getting stressed by something and can suggest certain types of supplements or even ask you to be mindful and think of your happy place. By using these techniques (among others previously mentioned), the present invention will be able to improve both the mental and physical health of users. In the future, the real-time tracking of DNA and protein synthesis will further allow the app and AI to test and track data for the user. Structures like this do exist but will be pushed further in the present invention with the use of AI. In the present invention, the AI will be able to use hormone sensors and future DNA and protein synthesis monitors to create a holistic AI health program for the user. Through the hormone tests and other tests including the use of vagus nerve stimulators, the AI will be able to track changes made to the user's DNA, mind, and wholistic version of sensing and promoting the body's best state to suggest further therapies and treatments with overall functionality and to either give the desired effect or an end result, basing results on overall happiness in life through different therapies and suggested experiences. These therapies can include hormone release pumps or suggested food, supplements, drugs, exercise, cranial electrostimulation, and vagus nerve stimulation, among others. With the real-time tracking of DNA sensors and vagus nerve stimulators, the present invention will be able to constantly monitor how the user reacts to these stimulators in ways that therapists cannot. With the addition of Radio Frequency Identification (RFID) supplements and drugs, the app will be able to keep track of all drugs and supplements the user is taking in a much more convenient way than a therapist or doctor can.

After the best therapeutic techniques are identified, a reference point can track the progress of the user. From there, deeper questions will be asked to see what is bothering the user. These questions will be like a typical therapy session or maybe visual or auditory stimulation that has some videos or music containing certain meanings behind it based on the user's biometrics and mental states. You could start diagnosing along with identifying what they said is bothering them, it could start to ask further questions. In this way, the AI behaves much like a fortune teller, who uses certain tells as an individual talks to them in order to tell their future or in this case, diagnose them. The AI is aware of the person's biometrics so it can ask certain questions based on these data and then go down another branch and another, till you get to a much closer diagnosis, and provide suggestive therapies or even suggest a therapist that could use the information to help get to the patient's issue faster and more thoroughly.

A comparable scenario in traditional therapy would be a case in which a therapist passes a particular problem to a specialist for a more human intervention if the issue is something that a human therapist can help with but done through AI gives the user the opportunity to be more vulnerable and thus go further in their therapy journey. While these questions are asked, the AI will track the user's facial expressions, micro-expressions, lie detection, etc. which will allow the AI to determine what is really bothering the user in ways that a typical human therapist cannot track. Based on the best therapeutic techniques for the user, the AI will be able to give the best treatment options for the user and can continuously track the progress that the user is making. In this way, the AI provides the user with 24-hour access to therapy, whereas a traditional therapist is only available to their patients an hour or so per week.

Another advantage of the present invention is its ability to track the user's productivity and performance in their work output and everyday life. Financial information can be provided to the AI, allowing it to assess the impact of the user's financial status on their overall wellbeing. This gives the present invention the ability to provide real-time feedback and alternative suggestions to the user should patterns of financial loss (perhaps due to a gambling addiction) be detected in order to treat the issue and prevent further stress and health decline. Furthermore, fingerprint scanning of the individual could provide the AI with an immediate and transparent background on the user, which will allow the AI to gain a better understanding of the individual, match them with similar datasets from other users, and quickly create treatment plans based on the information provided.

With the use of biofeedback, the AI can not only suggest the best possible treatment options for the user, but it can also help influence the future behaviors of user. Examples of some future treatment options the AI can give include the measuring of hormones to determine the patient's progress in real-time. With the use of a different sensor suite that can measure hormones in real-time, the level of certainty with the other normal-typical sensors along with a blood type with a good probability can be measured to figure out a person's internal chemical disposition within the given parameters. With the use of sensors such as the vagus nerve stimulator, there is additional cutting-edge technology that can be harnessed. For example, gently stimulating the vagal nerves will increase serotonin and lower cortisol, proving a successful treatment for patients if that is the therapeutic technique that works best for them. By testing how the user reacts to this stimulation, the AI can determine if the stimulation has a great effect on a user who needs to raise serotonin and lower cortisol levels.

Another one of these attached sensors can detect cortisol using engineered strands of DNA called aptamers, which are designed so that a cortisol molecule will fit into each aptamer, like how a key fits into a lock. When the cortisol attaches, the aptamer changes shape in a way that alters the electric fields at the surface of a transistor. With all these technologies and the following analysis in a database for each user, the amount of information from these sensors, along with other methods of testing can help predict the best form of therapy. It can also learn from multiple people with similar traits, traumas, and living conditions. Measuring input—output and finding the desired result based on the desired outcome brings the user to a flow state of happiness in a macro. As people typically can't be happy on a micro scale, sometimes seeing the work that they're doing may not make them happy at the moment, but measuring their levels of dopamine, a hormone that is released when you're motivated will suppress the micro discomfort.

This is the difference between the program and a therapist, the program has the ability to see that the user may not be happy on a micro level at the moment, but they are motivated by the potential outcome. This can be considered when a therapist cannot understand an overall picture of the full state of the body in just a few sessions. When the patient is experiencing tunnel vision due to a fight or flight response or trauma, this may be able to help them find some light and see in real-time when certain stimulation promotes the different motivational chemical releases, such as dopamine, to suggest a road to something the user enjoys. If the other things the AI suggests, like watching the reward chemicals, are healthy, based on what makes the user overall happy and productive, a situation could be argued that if a person must be productive to be happy, then typically that person does something, and they get a reward (or chemicals depending on what the user did). This could be motivating, being able to really see your own real-time information. This is about finding a balance in family, psychology, nutrition, supplements, productivity, actions, and experiences that can give the user an overall sense of happiness and can create continual live feedback to begin predicting the best pattern of therapies to deliver to the user to give them the fasts and best method to recover and keep them in an optimal place.

The AI system is safe and human-run and can be told when to turn off. If a user wants a break from the therapeutic techniques and wants to rest, the AI can be turned off in order to give the user complete control of the application. The AI will also have the ability to tell if the user is in need of a break from the therapeutic techniques and offer to give the user a break. The AI is noninvasive and driven by humans to achieve balance in health and wellness. The present invention could be implemented into such uses as dating, social applications, interrogation, human resources, copping skills, marketing, and access control. Any sort of use where very detailed and relevant data sets could help correlate data for diagnostics, statistics for research, pattern relevancy, analysis, and/or use where a very intricate data set of a person much more detailed than your average profile is used to match certain characteristics amongst other users to match them. The present invention could also be used as a security profile for a user for encryption. Most of this is with the users ok, to their data sets. Put another way, the present invention contains the ability to be applied beyond the world of wellness. Artificial intelligence, when connected to several biosensors as is the case in the present invention, has the ability to be used for security purposes as a screening device or other security devices. The present invention also offers key insights that could be used for marketing and human resources and other similar applications. In line with the wellness aspect of the present invention, it can also be applied as a lifestyle coach with applications in the world of socializing and dating and other aspects of day-to-day living.

Other features and aspects of the invention will become apparent from the following detailed description, taken in conjunction with the accompanying drawings, which illustrate, by way of example, the features in accordance with embodiments of the invention. The summary is not intended to limit the scope of the invention, which is defined solely by the claims attached hereto.

### BRIEF DESCRIPTION OF THE DRAWINGS

The various embodiments are illustrated by way of example, and not by way of limitation, in the figures of the accompanying drawings. Having thus described the invention in general terms, reference will now be made to the accompanying drawings, which are not necessarily drawn to scale, and wherein:

FIG. 1 is a flow diagram summary of the present invention.

FIG. 2 is a diagram showing the AI determining which therapeutic technique is best for a user.

FIG. 3 is diagram showing the AI's analysis of the user during the test of a therapeutic technique.

FIG. 4 is a diagram showing an example of a therapeutic technique the AI can use.

FIG. 5 is a diagram showing the AI asking the user deeper questions while analyzing the reactions from the user.

FIG. 6A-C are flow diagrams showing how the app uses different sensors and the data to find therapeutic techniques.

FIG. 7 is a flow diagram showing the different tests run by the app.

FIG. 8 is a diagram showing the Vagus nerve stimulation.

FIG. 9 is a diagram showing the inputs and outputs of two users.

FIG. 10 is a diagram showing how the user can take a break.

FIG. 11 is a line diagram illustrating a decentralized network.

FIG. 12 is a line diagram illustrating a distributed network.

FIG. 13 is a diagram showing the present invention's ability to contact emergency services.

FIG. 14 is a diagram outlining the flow of data collection in a secure database.

FIG. 15 is a flow diagram summarizing the integration of the vagus nerve stimulation therapy in the present invention.

FIG. 16A is a flow diagram summarizing possible scenarios in which the biosensors detect signs of stress and the AI attempts to suggest the best treatment.

FIG. 16B is a flow diagram outlining the flow of data from the biosensors to the cloud space via neural network encryption.

FIG. 17 is a diagram outlining the web services incorporated with server-client communication.

### DETAILED DESCRIPTION OF THE PREFERRED EMBODIMENT

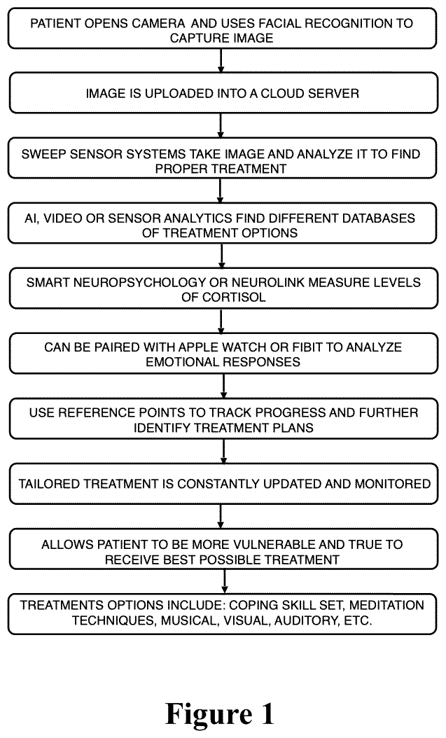

FIG. 1 is a flow diagram summary of the present invention. It begins with when the patient opens the camera and uses facial recognition to capture an image. Once the image is taken, it is uploaded into the cloud server. Once in the server, sweep sensor systems, like phone laser, phone camera, biometric sensors, outside camera, etc., take the image and analyze it to find proper treatment. AI, video or sensor analytics and databases analyze micro expressions, lie detection, eye movements, body language, and voice stress to lead into different databases of treatment options. Smart neuropsychology or neuro-link can measure cortisol levels to promote negative or positive feelings with psychological treatments. This can be paired with Smart Watches, Fitbit, and other devices to give biometrics through stress tests (EKG), pupil contraction through accessing memories, questions or videos shown to see emotional responses. This creates reference points, that are constantly updated and analyzed to track progress and further identify what treatment plans are right for the patient. This allows the patient to be completely vulnerable and true to find the best treatment option. These treatment options include coping skill set, meditation techniques, musical, visual, auditory, etc.

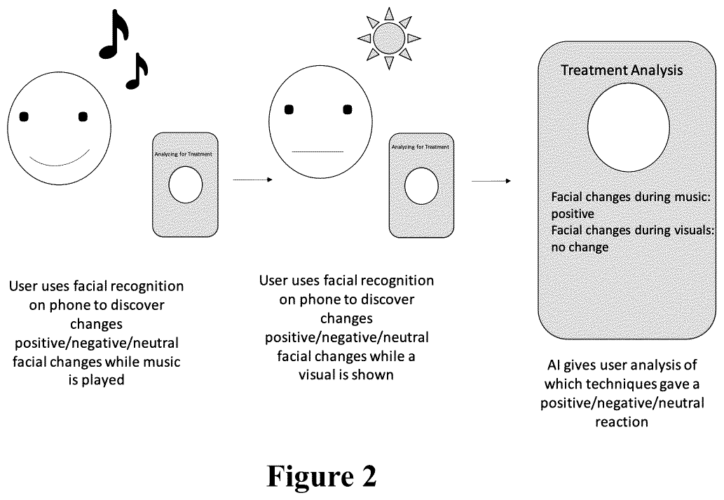

FIG. 2 is a diagram showing the AI determining which therapeutic technique is best for a user. In this diagram, the user is first listening to music, movies, or an AI therapist asking certain questions or talking to the user. While the user listens and/or watches the music, video, or person talking, mental stimulation the AI will use sweep sensors to track the user's reaction to the music, video, or person talking, mental stimulation and analyze the results of the reactions. After, the user will be shown positive images or given auditory feedback or visual based also on the user's reactions which can be recorded and predicated via eye movements. Examples of these eye movements are looking at cues to see if they look up and to a certain side, which hemisphere they're accessing while asked certain questions by visual cues or reactions. This process is like how interrogators ask base questions to see if someone is accessing certain hemispheres for auditory or visual thinkers to see if they are recalling a memory or making up a story. With this same process, the AI will be able to determine during the viewing and hearing of a movie, picture, music, or sounds how it makes the user feel and can give an analysis of whether their experience is something that can help the user or is something to be avoided. It is with this analysis that AI can determine the likes and dislikes of the user and how that can determine the best therapeutic techniques the user needs.

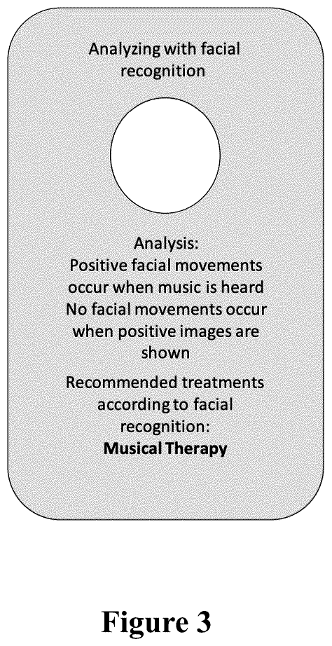

FIG. 3 is diagram showing the AI's analysis of the user during the test of a therapeutic technique. In this example, the user's reaction to music and visual images is being analyzed. The AI determines the user has positive or motivational facial movements while music is being played and no facial movements when visual images are shown. Because of the positive facial movements made while music is being played, the AI determines that musical therapeutic techniques would be the most beneficial for this user.

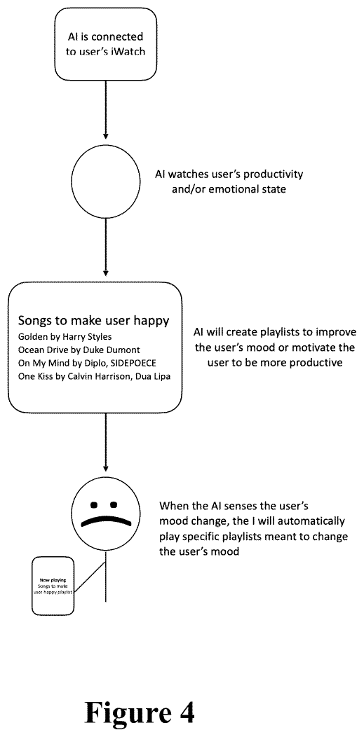

FIG. 4 is a diagram showing an example of AI using musical therapy technique. The AI can be linked to the user's laptop or iWatch to see if motivational music can be tailored to watch how productive the user is. The music or therapies can also be tailored to soothe the user, depending on if that is what is trying to be achieved. The AI can automatically suggest these things to the user so that the user can either become motivated or relax or to start to deal with some trauma. All these will be tailor fitted to the individual user so the user can experience the best therapeutic techniques. It will be as if the user has a therapist beside them to give them the musical motivation or relaxation they need at any time. The AI, with all the data collected, can also suggest based off a database of therapists what therapeutic techniques are the best and may even be able to send certain types of information to their therapist to guide them through the emotional distress and coping methods they may need. All these techniques and decisions will be determined by the approach the individual user wants to take. The user can also be given a suggestion on the best approach to take from the AI. The suggestions and techniques can be changed throughout time, depending on the timeline of the user's life, and other techniques may become a better fit as time progresses. Through the collection of all this information through the different therapeutic techniques and sensor data, the AI can determine if the user is progressing.

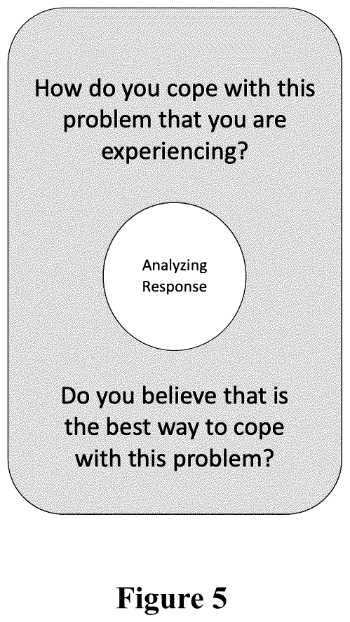

FIG. 5 is a diagram showing the AI asking the user deeper questions while analyzing the reactions from the user. The AI will ask the user a question like “how do you cope with the problem that you are experiencing?” The AI will watch the user's facial reactions and micro expressions etc. to determine the user's true feelings. The AI will then ask another question like “do you believe that is the best way to cope with this problem?” And based again on the facial and micro expressions, the AI can determine the user's true feelings. With these analyses and knowledge of the best therapeutic techniques for the user, the AI can give the best help to the user.

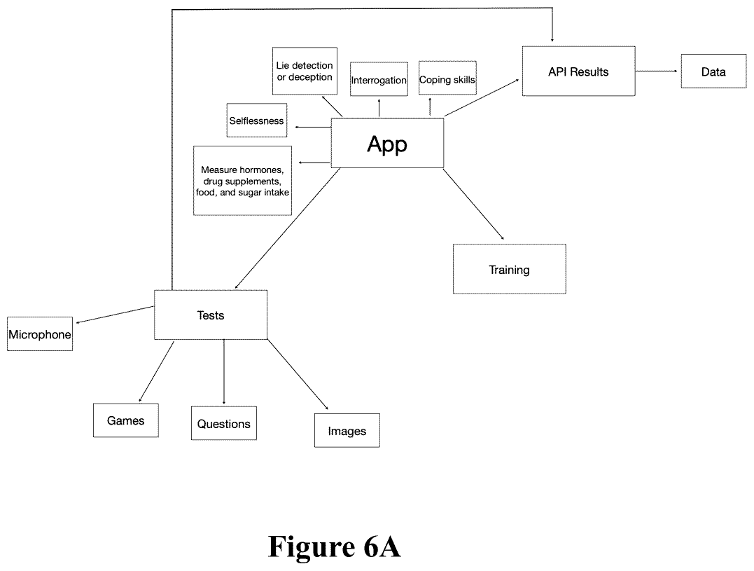

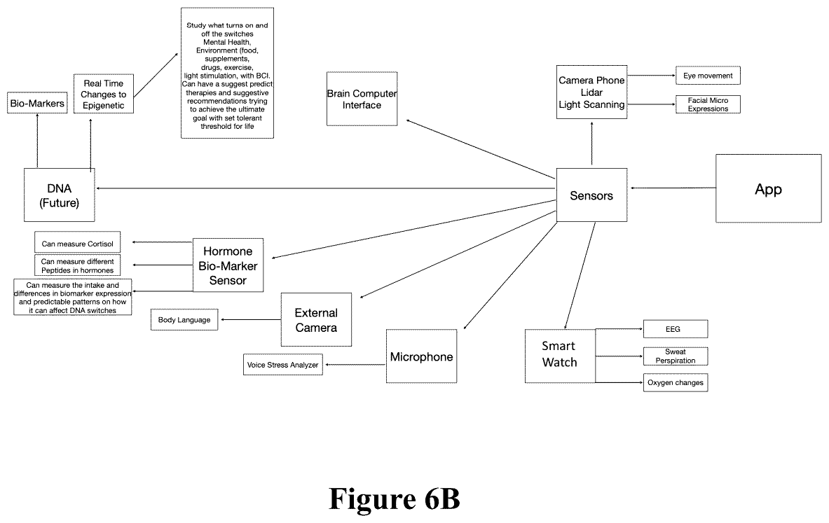

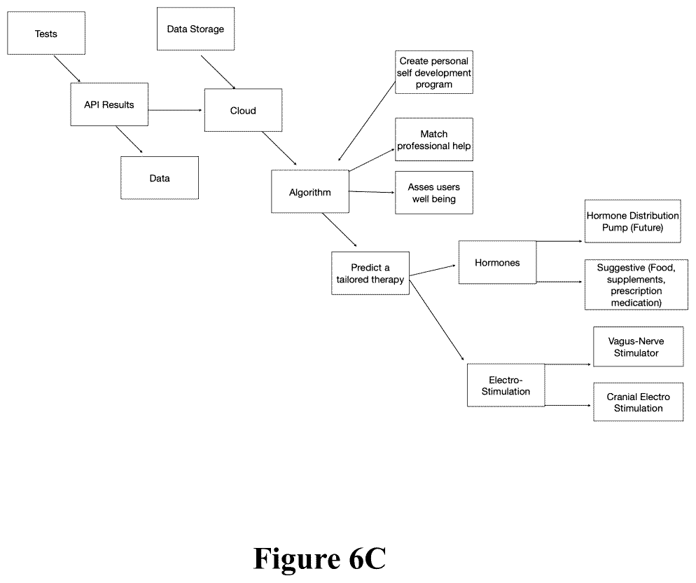

FIG. 6A is a flow diagram showing how the app uses different tests to gain results. The different tests the app uses are through images, questions, games, and the microphone. Through these tests that are run, the app will receive API results that give data to the app. The app can also use training through AI to learn different coping skills or other treatment options. The app can also be used as assistance for dating apps, coping skills, lie detection or deception, selflessness, measure hormones, drugs, supplement, food, and sugar intake and measure it to correlate it to the state of the user's wellbeing. All of these results are recorded as data.

FIG. 6B is a continuation of how the app uses sensors for the tests it runs. The app can connect to a Smart Watch and record EEG, sweat and perspiration, and oxygen changes to determine certain results. The app can use the microphone to run a voice stress analyzer test. With the external camera, the app can run tests on body language. With the hormone bio-marker sensor, the app can measure cortisol, different peptides in hormones, and measure the intake and differences in biomarker expressions and predictions on how it can affect DNA switches. With the camera phone, lidar, light scanning, the app can test eye movements and facial micro expressions. The app can also use the brain computer interface to run tests. In the future, the app can use DNA to run tests. These tests will use biomarkers and real time changes to Epigenetics, which will study what turns on and off switches in the mental health environment, such as food, supplements, drugs, exercise, light stimulation, and BCI. This can suggest and predict therapies and recommendations to achieve certain goals within a set tolerant threshold for life.

FIG. 6C is a continuation of the tests and data the app receives. Through the tests, the app will receive API results, which becomes data. The API results are stored in the cloud and allow for an algorithm to be created. Through the algorithm, the app can match the user to professional help, assess users' wellbeing, and predict a tailored therapy. The tailored therapy can be with hormones treatment, such as hormone distribution pump (in the future) or suggestive through food, supplements, or prescription medication. The tailored therapy can also be through electro-stimulation, such as Vagus nerve stimulation or cranial electro stimulation. The personal self-development program will also go back to the algorithm to continue constant learning by the app.

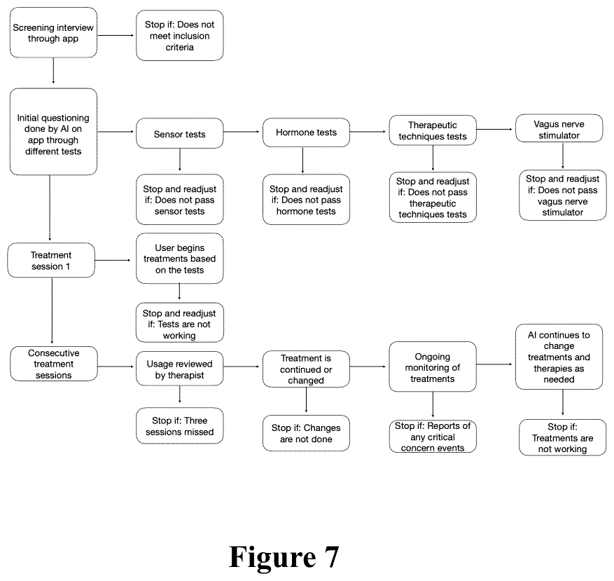

FIG. 7 is a flow diagram showing the different tests run by the app. The app begins with a screening of the user, and if that user does not fit the criteria, then the user cannot continue. After the initial screening interview, the initial questioning done on the app through the AI is done. These initial questioning tests are done through sensor testing, hormone testing, therapeutic techniques testing, and vagus nerve stimulators. Through these tests, the AI adapts and adjusts to the user's result to find the best treatments. If none of these tests are passed, the AI stops and readjusts treatments for the user. As treatments are continued, the AI continues to monitor the user's responses and continues to readjust as necessary. The data of the treatment will be sent to a therapist to continue the treatment or to change the treatment for the user.

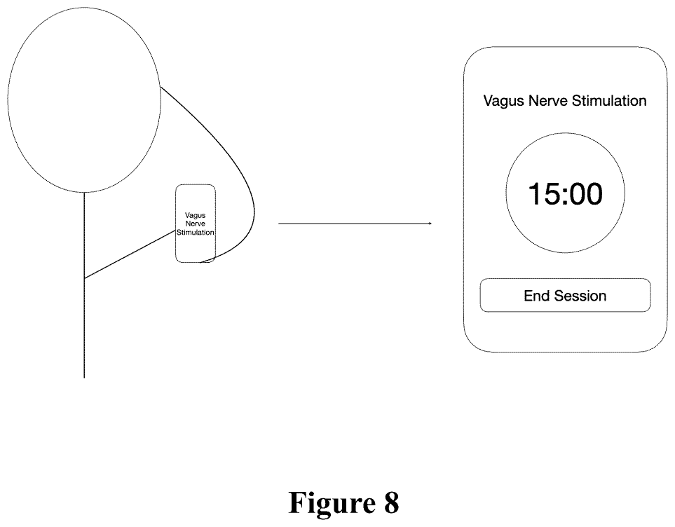

FIG. 8 is a diagram showing an example of how the Vagus nerve stimulation treatment can be used in the present invention. Like headphones, the stimulator can be plugged into the smartphone and attached to the head. The stimulation begins for a certain amount of time and is monitored by the AI to view the progress of the treatment.

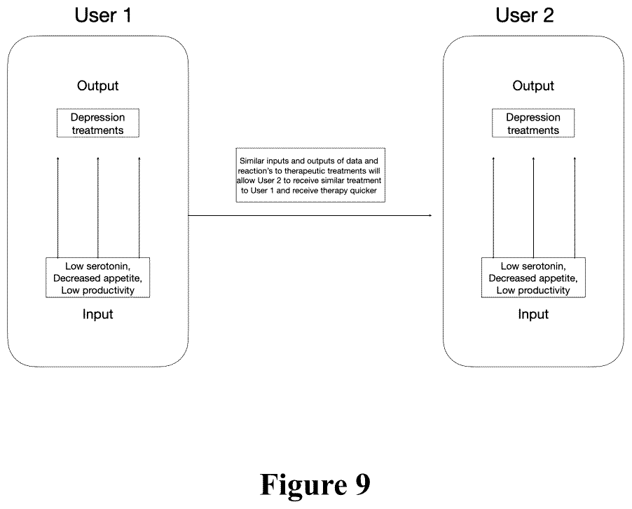

FIG. 9 is a diagram showing the inputs and outputs of two users. Both of these users have the same input and output. Because the present invention uses the ratios of the inputs and outputs to track changes in users, users with the same input and output ratios can receive the same treatment. This allows the second user to receive treatment plans quicker because user 1 has already received the treatments and has been successful with those treatments.

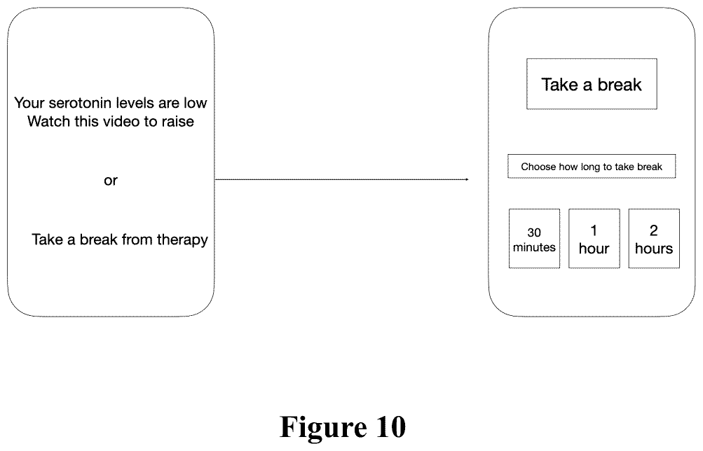

FIG. 10 is a diagram showing how the user can turn off the AI whenever the user needs a break. Because the user is in full control of the present invention, they have the ability to turn off the present invention at any time they need a break from treatment. The present invention can be turned off for however long the user needs it to be.

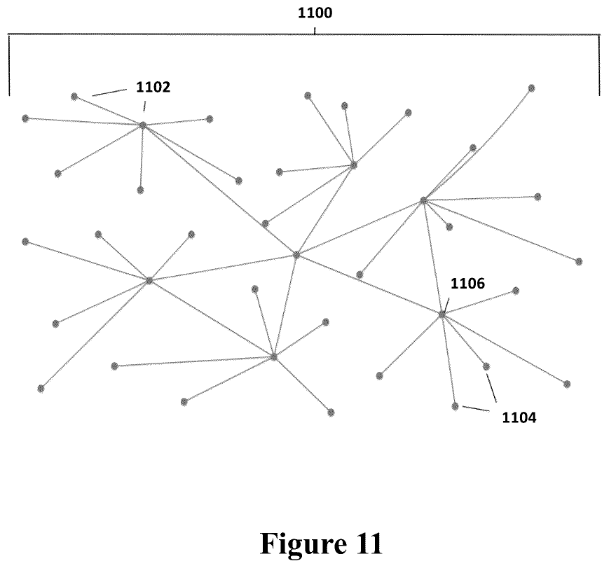

FIG. 11 is a line diagram illustrating a decentralized network. In accordance with the preferred embodiment of the present invention, the specific architecture of the network can be either decentralized or distributed. FIG. 11, generally represented by the numeral 1100, provides an illustrative diagram of the decentralized network. FIG. 11 depicts each node with a dot 1102 Under this system, each node is connected to at least one other node 1104. Only some nodes are connected to more than one node 1106.

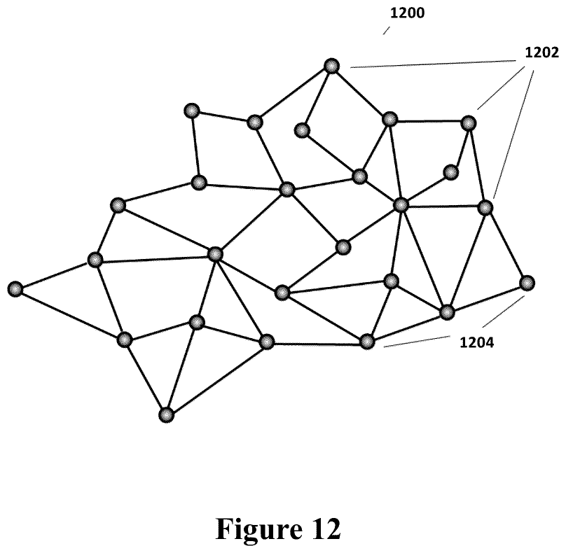

FIG. 12 is a line diagram illustrating a distributed network. For comparison purposes, FIG. 12, which is generally represented by the numeral 1200, illustrates a distributed network. Specifically, the illustration shows the interconnection of each node 1202 in a distributed decentralized network 1200. In accordance with the preferred embodiment of the present invention, each node 1202 in the distributed network 1200 is directly connected to at least two other nodes 1204. This allows each node 1202 to transact with at least one other node 1202 in the network. The present invention can be deployed on a centralized, decentralized, or distributed network.

In one embodiment, each transaction (or a block of transactions) is incorporated, confirmed, verified, included, or otherwise validated into the blockchain via a consensus protocol. Consensus is a dynamic method of reaching agreement regarding any transaction that occurs in a decentralized system. In one embodiment, a distributed hierarchical registry is provided for device discovery and communication. The distributed hierarchical registry comprises a plurality of registry groups at a first level of the hierarchical registry, each registry group comprising a plurality of registry servers. The plurality of registry servers in a registry group provide services comprising receiving client update information from client devices, and responding to client lookup requests from client devices. The plurality of registry servers in each of the plurality of registry groups provide the services using, at least in part, a quorum consensus protocol.

As another example, a method is provided for device discovery and communication using a distributed hierarchical registry. The method comprises broadcasting a request to identify a registry server, receiving a response from a registry server, and sending client update information to the registry server. The registry server is part of a registry group of the distributed hierarchical registry, and the registry group comprises a plurality of registry servers. The registry server updates other registry servers of the registry group with the client update information using, at least in part, a quorum consensus protocol.

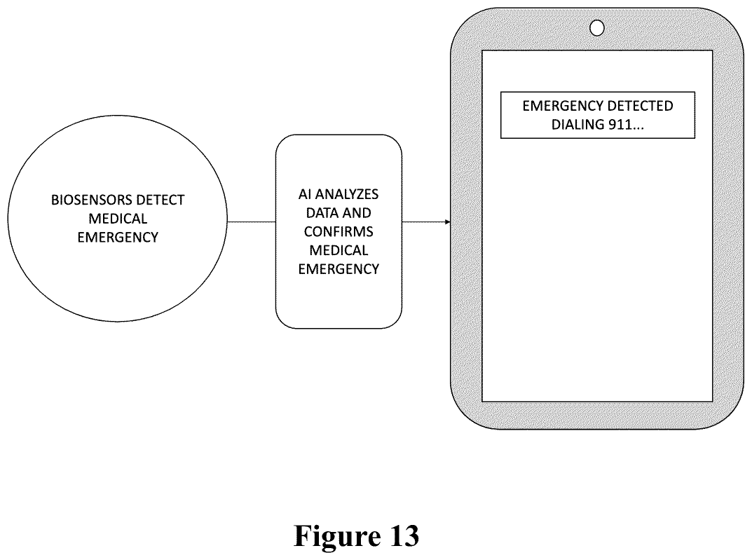

FIG. 13 is a diagram illustrating the ability of the present invention to contact emergency medical services when a medical emergency is detected. In accordance with the preferred embodiment, the present invention contains the ability to monitor several health biomarkers and analyze the recorded data in order to identify any patterns or abnormalities. Should AI determine that there is a severe abnormality in a given biometric recorded from any of the biosensors associated with the AI, the present invention can contact emergency services such as dialing 911 for the user before the abnormality becomes lethal, thus increasing the chances of survival for a given health emergency such as heart attacks, seizures, and others. Prior to making the call, a warning will be displayed to the user, informing them of the detected situation. In this moment, the user can choose to disable the emergency call or proceed in order to decrease the chances of a false call to an emergency service.

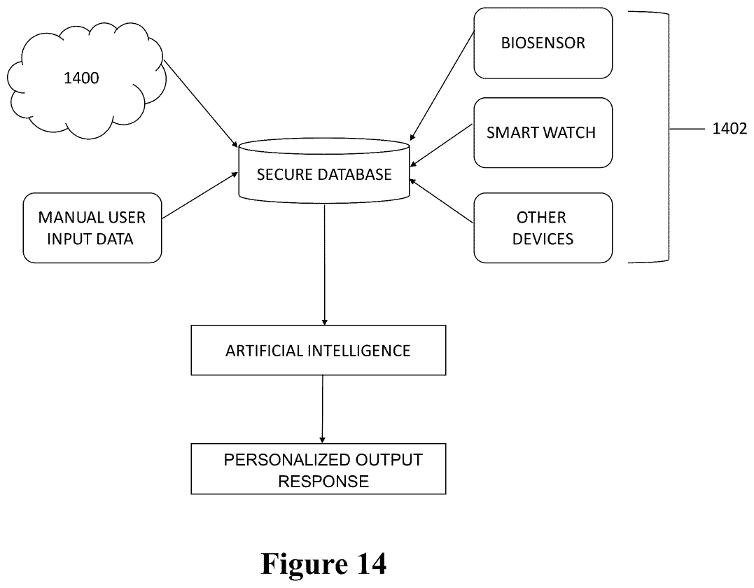

FIG. 14 is a diagram outlining the several sources of data collection which is transmitted to a secure database where AI can access the data in order to provide the user with personalized suggestions regarding their health, lifestyle, social life, and more. In accordance with the preferred embodiment, sources 1402 provide data to a secure database. These sources can be smart watches, smart scales, and other biosensors and devices. Data regarding the user's health and wellness is collected and transmitted from these devices to a secure database which is accessible to AI. The AI analyzes these data from the user along with data across multiple users provided via cloud, blockchain, or other secure or encrypted forms of data transmission 1400. The data analysis enables AI to determine the best possible treatment to provide to the user or the best fitted response to a given prompt provided by the user. Data is also collected from the user who may choose to manually input data onto their wireless mobile device to provide additional information beyond that recorded by the biosensors, smart watch, and other devices.

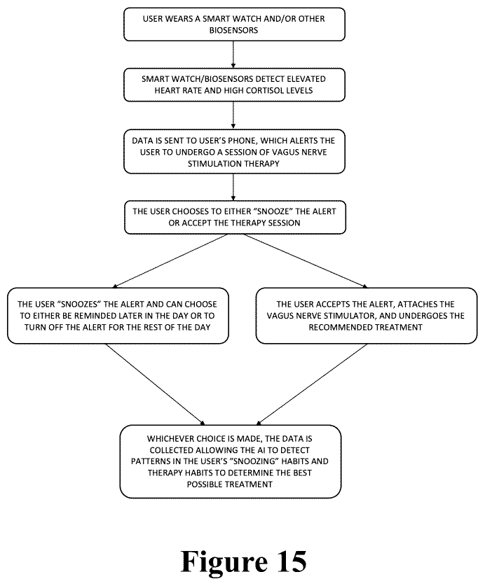

FIG. 15 is a flow diagram outlining a possible scenario in which vagus nerve stimulation therapy is integrated into the present invention. In accordance with the preferred embodiment, the user's biosensors/smart devices detect a stress response (increased heart rate, high cortisol levels, etc.) and send this feedback to the AI. Based on previous patterns, the AI will then determine if it should alert the user to partake in vagus nerve stimulation therapy. The user can then decide to either “snooze” this alert for later in the day, cancel the alert for the entire day, or accept the alert and begin vagus nerve stimulation therapy for the suggested amount of time. The AI then records the response of the user and learns from this data as to whether these alerts are helpful given the time of day, the location, etc. In which the alert was given and determine if the treatment is productive for the user based on biofeedback collected during the stimulation session.

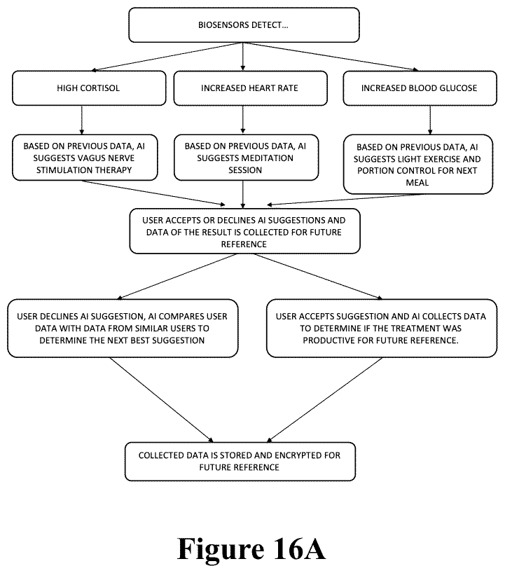

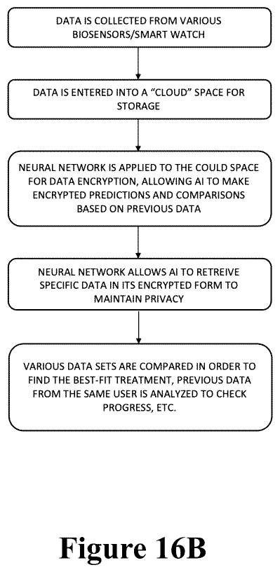

FIG. 16A is a flow diagram outlining a possible scenario in which the biosensors detect signs of stress. In accordance with the preferred embodiment, the biosensors detect a fluctuation in the user's resting state that indicates stress (high cortisol levels, increased heart rate, increased blood glucose, etc.) which is followed by the AI's response. This response is an attempt to suggest the best-fit treatment based on data collected from the user and previous users with similar profiles. The user is then allowed to decide whether or not they will accept the suggested treatment. Whichever they choose, data will be collected by the AI system to use as reference when the same signs of stress are detected in the future. If the user chooses to decline the treatment, the AI will search the database for similar profiles and choose the next best-fit suggestion. If the user chooses to accept the treatment, the AI will record via biosensors how the user responds to the treatment and if the treatment is successful, the AI will store this information and repeat the same treatment when the stress signs occur again.

FIG. 16B is a flow diagram showing the flow of data from the biosensor collection to the cloud space encryption. In accordance with the preferred embodiment, data is collected from the user's Smart Watch, various biosensors, and wearable medical devices. This data is then uploaded to a cloud space integrated with neural networks that encrypt the data. The AI is able to use the neural network encryption to compare and predict data within the database while maintaining encryption. This allows the data from all users to be accessed by the AI with total privacy.

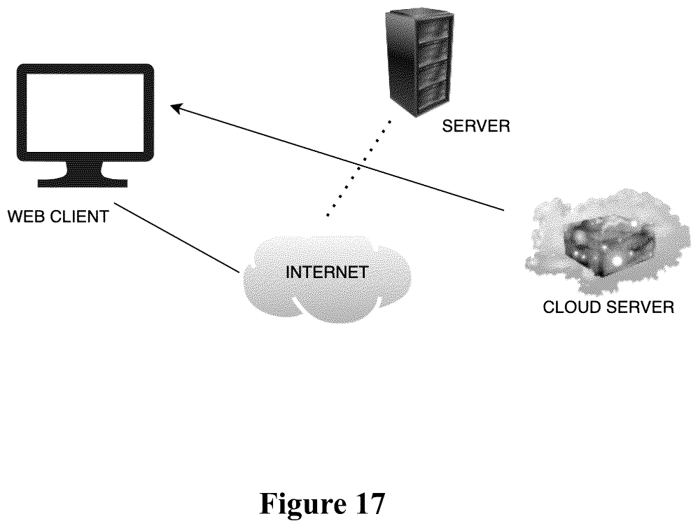

FIG. 17 is a diagram outlining the role of web services in the present invention. In accordance with the preferred embodiment, a web client interacts with the server ecosystem by way of a service connection, such as the internet, which then distributes data and pertinent information such as the web service platform to the cloud server and preliminary servers. This allows for data to be streamlined between the client and the server as well as cloud servers and other database systems. Communication between web services may be completed via Simple Object Access Protocol (SOAP) which allows multiple web service applications to communicate rapidly and efficiently and to provide data to the web client.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that may be included in the disclosed technology. The disclosed technology is not restricted to the illustrated example architectures or configurations, but the desired features may be implemented using a variety of alternative architectures and configurations. Indeed, it will be apparent to one of skill in the art how alternative functional, logical or physical partitioning and configurations may be implemented to implement the desired features of the technology disclosed herein. Also, a multitude of different constituent module names other than those depicted herein may be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

What is claimed is:

1. A wellness promoting system comprising:
a plurality of sensors configured to collect real-time physiological and mental state data from a user of said system;
a plurality of databases configured to receive and store said real-time physiological and mental state the plurality of said databases comprising user specific databases and non-user-specific databases;
at least one user device containing at least one camera and at least one microphone wherein said wellness promoting system continuously accesses said at least one camera to collect real-time facial expression data including body language and emotional state and at least one microphone to collect voice stress data, said at least one user device further configured to collect haptic data including touch and pressure sensitivity; and
a wellness biometric monitor configured to identify signatures across all of said plurality of sensors and predict a best form of therapy via artificial intelligence analysis provided via one or more processors, wherein said one or more processors are configured to receive said real-time physiological and mental state data, real-time facial expression data, said haptic data, said voice stress data, and non-real time information, process said real-time physiological and mental state data to obtain a plurality of physiological and mental state wellness parameters, and wherein said artificial intelligence is a neural network trained to identify an optimal treatment plan for a user via analyzing and analyze said real-time physiological and mental state data, said facial expression data, said haptic data, said voice stress data, and said non-real time information and generating a plurality of personalized wellness suggestions based on said analysis for said user provided via said at least one user device wherein said personalized wellness suggestions are determined via said artificial intelligence analysis of said physiological and mental state data, said facial expression data, said haptic data, said voice stress data, and said non-real time information and are tailored to said user in order to promote physiological and mental wellness, and output of said wellness monitor based at least in part on one or more features of said plurality of physiological and mental state parameters and said treatment plan and store said one or more features in a memory of said wellness monitor, wherein said artificial intelligence is further configured to compare prior user data with real-time user data, and compare both of said prior user data and real-time user data against non-user data, so that said user is provided information as to a state of present wellness and provided with a plurality of recommendations to improve a current wellness of said user, and wherein said recommendations are provided to said user via alerts on said at least one user device, wherein said wellness monitor is further configured to detect based on analysis of said real-time user data, emergency health events, provide said user with alerts regarding said emergency health event, and calls emergency responders, and wherein said wellness biometric monitor is further configured to compare data across a plurality of users in order to identify patterns across said plurality of users.

2. (canceled)

3. (canceled)

4. The system of claim 1, wherein an intake of medication and supplements by said user is monitored via RFID.

5. The system of claim 4 wherein information regarding said intake of medication and supplements is relayed to said wellness monitor wherein said information regarding said intake of medication and supplements is analyzed and stored for future reference.

6. The system of claim 1, wherein user-specific data stored within said plurality of databases includes family history, genealogy, genetic information, environmental information, financial information, career information, and lifestyle information of said user, and wherein said user-specific data is analyzed via artificial intelligence to determine a wellness plan for said user.

7. The system of claim 1, wherein said non-real time information comprises databases in communication with said wellness monitor and said plurality of sensors via a data network.

8. A method for promoting wellness comprising:
a user activating a plurality of sensors configured to generate real-time physiological and mental state data from said user;
accessing a plurality of databases configured to receive and store said real-time physiological and mental state data relevant to a wellness assessment, the plurality of said databases comprising user specific databases and non-user-specific databases;
at least one user device containing at least one camera and at least one microphone wherein a wellness promoting system continuously accesses said at least one camera to collect real-time facial expression data including eye movement tracking, micro facial expressions, body language and emotional state and at least one microphone to collect voice stress data, said at least one user device further configured to collect haptic data including touch and pressure sensitivity; and
accessing a wellness biometric monitor configured to look for signatures, wherein said biometric monitor is paired along with other sensors and is accessible to an artificial intelligence program configured to predict a best form of therapy via one or more processors, wherein said one or more processors are configured to receive said real-time physiological and mental state data, said real-time facial expression data, said haptic data, said voice stress data, and non-real time information, process said real-time physiological and mental state data to obtain a plurality of physiological and mental state wellness parameters, analyze real-time physiological and mental state data, said facial expression data, said haptic data, said voice stress data, and said non-real time information, and process said real-time physiological and mental state data, said real-time facial expression data, said haptic data, said voice stress data, and non-real time information, process said real-time physiological and mental state data to obtain a plurality of physiological and mental state wellness parameters, and wherein said artificial intelligence program is a neural network trained to identify an optimal treatment plan for a user via analyzing said real-time physiological and mental state data, said facial expression data, said haptic data, said voice stress data, and said non-real time information and generating a plurality of personalized wellness suggestions for said user wherein said personalized wellness suggestions are tailored to said user based on said analysis in order to promote physiological and mental wellness and prevent an onset of illness or other physiological or mental health conditions, and output of said wellness monitor based at least in part on one or more features of said plurality of physiological and mental state parameters and said treatment plan and store said one or more features in a memory of said wellness monitor, wherein said artificial intelligence compares prior user data with real-time user data, and compare both of said prior user data and real-time user data against non-user data, so that said user is provided information as to a state of present wellness and provided with a plurality of recommendations to improve current wellness, and wherein said recommendations are provided to said user via alerts and on said at least one user device, and wherein said wellness biometric monitor is further configured to compare data across a plurality of users in order to identify patterns across said plurality of users.

9. (canceled)

10. The method of claim 8, wherein said plurality of databases comprise a user's medical history and medical records as reported by said user or by a primary care provider of said user and wherein said method further comprises redirecting information to said primary care provider of said user.

11. The method of claim 8, wherein an intake of medication and supplements by said user is monitored via RFID.

12. The method of claim 11 wherein information regarding an intake of medication and supplements by said user is relayed to said wellness monitor wherein said information regarding said intake of medication and supplements is analyzed and stored for future reference.

13. The method of claim 8, wherein said plurality of databases further comprise a family history, genetic information, environmental information, financial information, career information, and lifestyle information of said user and wherein said artificial intelligence accesses said plurality of databases for user-specific analysis.

14. The method of claim 8, wherein said non-real time information comprises databases in communication with said wellness monitor via a data network.

15. The method of claim 8, wherein said physiological data indicates a stress level of said user and informs said wellness monitor to generate real-time wellness suggestions to decrease said stress level of said user.

16. The method of claim 8, wherein said recommendation includes suggestions for vagus nerve stimulation therapy sessions and wherein said wellness monitor collects physiological and mental data before, during, and after said vagus nerve stimulation therapy sessions in order to determine an efficacy of said vagus nerve stimulation therapy.

17. The method of claim 8, wherein said recommendation includes suggestions for said user to listen to third-party audio as determined by artificial intelligence analysis of a prior listening history of said user and said user's physiological and mental state associated with said prior listening history.

18. The method of claim 8, wherein said recommendation includes suggestions for said user to partake in a given method of therapy as determined by artificial intelligence analysis of a user's prior therapy history and said user's physiological and mental state associated with said prior therapy history.

19. The method of claim 18, wherein said artificial intelligence analyzes real-time physiological data during said suggested therapy in order to determine whether said suggested therapy is physiologically beneficial to said user and uses said real-time physiological data to inform future therapy suggestions.

20. The method of claim 8, wherein said artificial intelligence can access data across third-party social media applications and generate social media usage recommendations based on analysis of said social media applications in order to increase an overall wellness for said user.

21. The method of claim 8, wherein said recommendation includes suggestions regarding potential healthy coping skills, methods for developing healthy coping strategies and artificial intelligence analysis of a progress of said user to determine which said suggested potential healthy coping skills are beneficial.

22. The method of claim 8, wherein said wellness promoting system detects concerning changes in said user's real-time physiological and mental data and calls medical professionals to assist said user during a physiological or mental health crisis for immediate intervention and emergency care.

23. The method of claim 8, wherein said sensors include molecular sensors able to monitor and detect harmful hormonal changes to molecular health and DNA structure of said user, and wherein said method further comprises a step wherein said user is notified if hormonal changes are detected and provided with updated suggestions for hormonal optimization.

24. (canceled)

25. (canceled)

26. (canceled)

27. The method of claim 8, wherein facial expression monitoring is tracked to determine a mood of said user and response to said treatment plan.

28. A method for promoting wellness comprising:
a user activating a plurality of sensors configured to generate real-time physiological and mental state data from said user;
accessing a plurality of databases configured to receive and store said real-time physiological and mental state data relevant to a wellness assessment, the plurality of said databases comprising user specific databases and non-user-specific databases;
accessing at least one user device containing at least one camera and at least one microphone wherein a wellness promoting system continuously accesses said at least one camera to collect real-time facial expression data including eye movement tracking, micro facial expressions, body language and emotional state and at least one microphone to collect voice stress data, said at least one user device further configured to collect haptic data including touch and pressure sensitivity; and
accessing a wellness biometric monitor configured to look for signatures and predict a best form of therapy via pairing with other sensors and an artificial intelligence program via one or more processors, wherein said one or more processors are configured to receive said real-time physiological and mental state data and non-real time information, process said real-time physiological and mental state data to obtain a plurality of physiological and mental state wellness parameters, and wherein said artificial intelligence program is a neural network trained to identify an optimal treatment plan for a user via analyzing said real-time physiological and mental state data, said facial expression data, said haptic data, said voice stress data, and said non-real time information, and wherein personalized wellness suggestions are determined via said artificial intelligence and are tailored to said user in order to promote physiological and mental wellness and prevent an onset of illness or other physiological or mental health conditions, and process said information to generate a treatment plan said wellness monitor further configured to modify content of a display and an audio transducer perceptible by said user, so that modulation is controlled to promote user wellness and recommendations are provided to said user via alerts on said at least one user device, and wherein said wellness monitor is further configured to detect based on analysis of said real-time user data, emergency health events, provide said user with alerts regarding said emergency health event, and calls emergency responders, and wherein said wellness biometric monitor is further configured to compare data across a plurality of users in order to identify patterns across said plurality of users.

29. The method of claim 28, wherein said wellness monitor is granted access to a camera on said at least one user device belonging to said user for real-time facial expression monitoring while said user uses said at least one user device.

30. The method of claim 28, wherein said wellness monitor is granted access to a microphone on said at least one user device belonging to said user for monitoring sounds proximate to said user in real-time.
